(19) United States
(12) Patent Application Publication
(10) Pub. No.: US 2008/0221530 A1
(43) Pub. Date: Sep. 11, 2008

(54) Injection Device With A GearBox

(75) Inventors: Kristian Glejbol, Glostrup (DK); Claus Schmidt Moller, Fredensborg (DK); Michael Ejstrup Hansen, Veflinge (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/911,869

(22) PCT Filed: Apr. 21, 2006
(86) PCT No.: PCT/EP2006/061747

§ 371 Date: May 21, 2008

(30) Foreign Application Priority Data
Apr. 24, 2005 (DK) PA 2005 00588

(51) Int. Cl. A61M 5/315; A61M 5/00; A61M 5/24

(52) U.S. Cl. 604/211; 604/208; 604/224; 222/390

(57) ABSTRACT

An injection device for injection of set dose of liquid medicine from a cartridge has a piston rod (4, 104)/nut (3, 113) assembly where a nut is screwed up along a threaded piston rod during a dose setting operation. During administration of the set dose the nut is also screwed along the piston rod, thus making the piston rod/nut assembly either longer or shorter. During dose setting the nut is screwed along the piston rod by rotating a dose setting drum (17). An injection button is coupled to the piston rod with a gear wheel transmission (9, 109). Movement of the injection button causes a corresponding movement of the piston rod until the nut engages an abutment in the housing (1).

The present invention relates to syringes having a piston drive element for engaging and moving a piston within a medication cartridge, by which a dose can be set by rotating a nut along the piston drive element during dose setting, and wherein a set dose can be expelled by driving the nut to its initial position.

### BACKGROUND OF THE INVENTION

An almost classic pen which adopts the above described dose setting and injecting scheme is described in EP 327 910.

An almost classic pen of this type is described in EP 327 910.

By setting a dose on this pen a tubular member forming an injection button is screwed up along a threaded piston rod a distance corresponding to the distance said piston rod must be moved to inject the set dose. The tubular member simply forms a nut which is during the dose setting screwed away form a stop and which is during the injection pressed back to abutment with said stop and the force exerted on the button is directly transmitted to the a piston closing one end of an ampoule in the syringe which ampoule contains the medicament to be injected. When the piston is pressed into the ampoule the medicament is pressed out through a needle mounted through a closure at the other end of the ampoule.

By time it has been wanted to store larger amount in the ampoules, typically 3 ml instead of 1.5 ml. As it has not been appropriate to make the syringe longer the ampoule is instead given a larger diameter, i.e. the area of the piston facing the medicament in the ampoule has been doubled and consequently the force which has to be exerted on the piston to provide the same pressure as previously inside the ampoule has been doubled. Further the distance the piston has to be moved to inject one unit of the medicament has been halved.

This development is not quite favourable, as especially users having reduced finger strength have their difficulties in pressing the injection button, a problem that is further increased when still thinner needles are used to reduce the pain by injection. Also with quite small movements of the button it is difficult to feel whether the button is moved at all and by injection of one unit from a 3 ml ampoule the piston and consequently the injection button has to be moved only about 0.1 mm.

Consequently a wish for a gearing between the injection button and the piston has occurred so that the button has a larger stroke than has the piston. By such a gearing the movement of the injection button is made larger and the force, which has to be exerted on the injection button, is correspondingly reduced.

In EP 608 343 a gearing is obtained by the fact that a dose setting element is screwed up along a spindle having a thread with a high pitch. When said dose setting element is pressed back in its axial direction the thread will induce a rotation of said dose setting element, which rotation is via a coupling transmitted to a driver nut with a fine pitch which driver nut will force a threaded not rotatable piston rod forward.

A similar gearing is provided in WO 99/38554 wherein the thread with the high pitch is cut in the outer surface of a dose setting drum and is engaged by a mating thread on the inner side of the cylindrical housing. However, by this kind of gearing relative large surfaces are sliding over each other so that most of the transformed force is lost due to friction between the sliding surfaces. Therefore a traditional gearing using mutual engaging gear wheels and racks is preferred.

From WO 96/26754 is known an injection device wherein two integrated gear wheels engages a rack fixed in the housing and a rack inside a plunger, respectively. When the plunger is moved axially in the housing the rack inside this plunger can drive the first gear wheel to make the other integral gear wheel move along the fixed rack in the housing. Thereby the gear wheel is moved in the direction of the plunger movement but a shorter distance than is this plunger and this axial movement of the integrated gear wheels is via a housing encompassing said gear wheels transmitted to a piston rod which presses the piston of an ampoule further into this ampoule. However, the rack inside the plunger is one of a number axial racks provided inside said plunger. These racks alternates with untoothed recesses, which allow axial movement of the plunger without the first gear wheel being in engagement with a rack in this plunger. This arrangement is provided to allow the plunger to be moved in a direction out of the housing when a dose is set. When the plunger is rotated to set a dose it is moved outward a distance corresponding to one unit during the part of the rotation where the first gear wheel passes the untoothed recess, thereafter the first gear wheel engages one of the racks so the set unit can be injected, or the rotation can be continued to make the first gear wheel pass the next recess during which passing the set dose is increased by one more unit and so on until a dose with the wanted number of units is set.

A disadvantage by this construction is that the teeth of the racks and gearwheels alternating have to be brought in and out of engagement with each other with the inherit danger of clashing. As only a few racks separated by intermediary untoothed recess can be placed along the inner surface of the plunger only few increments can be made during a 360° rotation.

In U.S. Pat. No. 6,663,602 is disclosed an injection device wherein a direct gearing, i.e. a gearing by which more transformations of rotational movement to linear movement and linear movement to rotational movement are avoided, between the injection button and the piston rod.

The injection device shown in U.S. Pat. No. 6,663,602 comprises a housing wherein a piston rod threaded with a first pitch is non rotatable but longitudinally displaceable guided, a nut engaging the thread of the piston rod which nut can be screwed along the threaded piston rod away from a defined position in the housing to set a dose and can be pressed back to said defined position carrying the piston rod with it when the set dose is injected, a dose setting member which can be screwed outward in the housing along a thread with a second pitch to lift an injection button with it up from the proximal end of the housing. This injection device is provided with a gearbox which provides a gearing between the axial movements of the injection button and the nut relative to the housing which gearing has a gearing ratio corresponding to the ratio of said second and first pitch.

Although broadly being considered state of the art, the injection device disclosed in U.S. Pat. No. 6,663,602 presents important mechanical constraints due to the construction principle of the linear gearing system which acts during dosing. According to the injection device of U.S. Pat. No. 6,663,602, the length of the nut/piston rod assembly is increased during the dose setting by rotating the nut in a thread on the piston rod. During dosing, the linear displacement of the nut and the injection button are coupled such that there is proportionality between the movement of the nut and the injection button, the proportionality given by the transmission ratio of the gear box. As the nut is rotationally confined relatively to the piston rod during dosing, the translation of the nut corresponds to the translation of the piston rod and thus ultimately to the dose of medication administered.

As the gear is an integrated part of the construction and as the gear can only be designed to certain transmission ratios the gear implies important constraints on the obtainable ratio of movement of the injection button relative to the movement of the piston. Due to the direct proportionality between the movement of the piston rod and the administered dose of medication, the same syringe can not easily be adapted to different medications. Although adaptation is possible by changing the transmission ratio of the linear gear, this solution will in general be very complex and imply a complete redesign of the syringe.

### DISCLOSURE OF THE INVENTION

It is the objective of the present invention to devise a novel mechanical feature of mechanically actuated syringes involving a linear transmission mechanism that provides an easy adjustment of the movement of the piston rod relatively to the linear movement of the dose setting button.

According to a first aspect of the present invention, the injection device comprises:

a. a housing having a proximal end and a distal end,

b. a threaded piston rod having a first thread with a first pitch and a second thread with a second pitch, the piston rod being linearly displaceable in the housing,

c. a member secured against rotational and axial motion with respect to the housing, the member forming a central threaded opening for engaging the second thread of piston rod;

d. a displaceable nut that moves relative to the housing, the nut engaging the first thread of the piston rod so that the nut can screw along the first thread of the piston rod, thereby being capable of moving along the piston rod from a first position on the piston rod to a second position on the piston rod during dose setting, and from the second to a third position during dosing, the displacement of the nut along the piston rod from the first position to the third position defining the quantity of medication expelled by the injection device;

e. displacement coupling means for coupling linear displacement of an injection button to the nut for injecting a set dose, and

f. means for preventing rotation of the nut when linear force is exerted on the injection button.

This injection device provides a piston rod that is rotated relatively to the nut during dose setting as well as during the dose operation. In rotation of the piston rod during dosing, the length of the piston rod/nut assembly is modified during administration of the dose. This length modification overlays the pure linear translation imposed on the piston rod/nut assembly by the gear.

By changing the length of the assembly during dosing, the administered dose is no longer fixed to the translation imposed by the movement of the injection button and gear ratio. Thus, the linear movement of the piston rod is now decoupled from the movement of the nut.

This is highly advantageous as a general syringe can be customized to specific drugs simply by determining how much the piston rod is rotated or counter rotated during dose administration.

According to a second aspect of the invention, the length of the nut/piston rod assembly is shortened during dosing and thus reducing the effective movement of the piston rod and increasing the precision by which the medication is delivered.

In one specific embodiment, the piston rod is counter rotated during dose administration thus reducing the amount of medication administered. This specific embodiment provides the possibility of providing an injection device having half-incremental dose setting by only substituting a minor number of components compared to the corresponding full-incremental injection device.

According to a third aspect of the invention, the length of the nut/piston rod assembly is prolonged during dosing, thus increasing the effective movement of the piston rod.

According to a fourth aspect of the invention, the pitches of the threaded parts and the linear gearing are matched such that the piston rod is essentially fixed relatively to the cartridge during dose setting.

According to a fifth aspect of the invention, the pitches of the threaded parts and the linear gearing are matched such that the piston rod retracts slightly relatively to the cartridge during dose setting.

According to a sixth aspect of the invention, the pitches of the threaded parts and the linear gearing are matched such that the piston rod retracts between 0% and 2% of the full linear translation relatively to the cartridge during dose setting. According to a further aspect of the invention, the gearing between the movements of the injection button and the nut is obtained by the gearbox comprising at least one gear wheel carried by a connector which projects from the gear box longitudinally displaceable but non rotatable relative to said gearbox and is integral with the nut, a first rack integral with a first element of the gearbox, which element is rotational but not longitudinally displaceable relative to the housing, and second element carrying a second rack projecting from said gearbox longitudinally displaceable but non rotatable relative to said first element and being coupled to the injection button to follow longitudinal movements of said button, the at least one gear wheel engaging the first and the second rack, respectively, and being dimensioned to provide a gearing by which a longitudinal movement of the second rack is transformed to a longitudinal movement of the connector with a gearing ratio for the mentioned longitudinal movements of the second rack and the connector relative to the housing, which gearing ratio corresponds to the ratio of said second to said first pitch.

In such a device only the forces necessary to drive the dose setting member are transformed by a thread with a high pitch whereas the forces necessary to move the piston by injection is transmitted to said piston through a conventional gear with constantly engaging gears and racks.

The piston rod may be provided with a stop for the movement of the nut along the thread of said piston rod. This way a dose setting limiter is provided in the classic way, which involves no additional members to prevent setting of a dose exceeding the amount of liquid left in the ampoule.

According to a still further aspect of the invention, the injection device comprises:

a. a housing having a proximal end and a distal end;

b. a piston rod having a longitudinal axis, the piston rod having a thread with a first pitch, the piston rod being linearly displaceable in the housing and being threadedly engaged with respect to the housing by means of a second thread on the piston rod,

c. a displaceable nut that moves relative to the housing, the nut engaging the first thread of the piston rod so that the nut can screw along the first thread of the piston rod, thereby being capable of moving along the piston rod in proximal direction from a first position on the piston rod to a second position on the piston rod,

d. a dose setting drum having an injection button disposed on an end thereof, the drum having a threaded surface with a third pitch, the dose setting drum engaging the housing and rotatable within the housing so that it may be screwed outward from the proximal end of the housing,

e. a gear box assembly coupling axial movement of the injection button with axial movement of the nut, the gear box providing a gearing having a ratio substantially corresponding to the ratio of the third pitch to the first pitch.

As used herein, the term “drug delivery device” according to the instant invention shall mean a single-dose or multi-dose, disposable or re-useable device designed to dispense a selected dose of a medicinal product, preferably multiple selected doses, e.g. insulin, growth hormones, low molecular weight heparins, and their analogues and/or derivatives etc. Said device may be of any shape, e.g. compact or pen-type. Dose delivery may be provided through a mechanical (optionally manual) or electrical drive mechanism or stored energy drive mechanism, such as a spring, etc. Dose selection may be provided through a manual mechanism or electronic mechanism. Additionally, said device may contain components designed to monitor physiological properties such as blood glucose levels, etc. Furthermore, the said device may comprise a needle or may be needle-free. In particular, preferred embodiment the term drug delivery device shall mean a disposable multi-dose pen-type device having mechanical and manual dose delivery and dose selection mechanisms, which is designed for regular use by persons without formal medical training such as patients. Preferably, the drug delivery device is of the injector-type.

The term “housing” according to instant invention shall preferably mean an exterior housing (“main housing”, “body”, “shell”) or interior housing (“inner body”, “insert”) having internal and external threads. The housing may be designed to enable the safe, correct, and comfortable handling of the drug delivery device or any of its mechanisms. Usually, it is designed to house, fix, protect, guide, and/or engage with any of the inner components of the drug delivery device (e.g., the drive mechanism, cartridge, plunger, piston rod) by limiting the exposure to contaminants, such as liquid, dust, dirt etc. In general, the housing may be unitary or a multipart component of tubular or non-tubular shape. Usually, the exterior housing serves to house a cartridge from which a number of doses of a medicinal product may by dispensed. However, a cartridge may be directly coupled to the housing so as to be partly or fully external to the housing.

The term “engaged” according to instant invention shall particularly mean the interlocking of two or more components of the drive mechanism/drug delivery device, e.g. a spline or thread connection, preferably the interlocking of helical threads of components (“threadedly engaged”). The term “engaged” encompasses the use of intermediary components coupling threadedly engaged threads.

The term “thread” according to instant invention shall preferably mean a full or part thread, e.g., a cylindrical spiral rib/groove, usually of helical nature or comprising linear inclined parts, located on the internal and/or external surface of a component of the drug delivery device (“internal thread” and/or “external thread”), having an essentially triangular or square or rounded section designed to allow continuous free rotational and/or axial movement between components. Optionally, a thread may be further designed to prevent rotational or axial movement of certain components in one direction.

The term “dose setting member” according to instant invention shall mean an essentially tubular component of essentially circular cross-section having either: a) both an internal and external thread, or b) an internal thread, or c) an external thread. The dose setting member may be designed to indicate a selected dose of a dispensable product. This may be achieved by use of markings, symbols, numerals, etc., e.g. printed on the external surface of the dose setting member or an odometer, or the like. Also, a separate dose scale may be used, either comprising a mechanical or an electronic display.

The term “lead” according to instant invention shall preferably mean the axial distance a nut would advance in one complete revolution; preferably “lead” shall mean the axial distance through which a component having a helical thread, i.e. dose setting member, nut, piston rod, etc., of the drive mechanism travels during one rotation. Therefore lead is a function of the pitch of the thread of the relevant component.

The term “pitch” according to instant invention shall preferably mean the distance between consecutive contours on a helical thread, measured parallel to the axis of the helical thread.

The term “piston rod” according to instant invention shall mean a component adapted to operate through/within the housing, designed to translate axial movement through/within the drug delivery device, preferably from the nut to the piston, for the purpose of discharging/dispensing an injectable product. Said piston rod may be flexible or not. It may be a simple rod, a lead-screw, or the like. The “piston rod” according to instant invention shall further mean a component having a non-circular cross-section and preferably having one or more threads located partly or fully extending along its length. It may be made of any suitable material known by a person skilled in the art.

The term “incremental feedback mechanism” according to instant invention shall mean a mechanism providing the dose setting to occur in a number of discrete incremental steps, e.g. one incremental click for each international unit being set, the incremental feedback mechanism preferably providing a tactile or audible click which can be sensed or heard by a user operating the pen.

In the following the invention is described in further details with references to the drawing, wherein

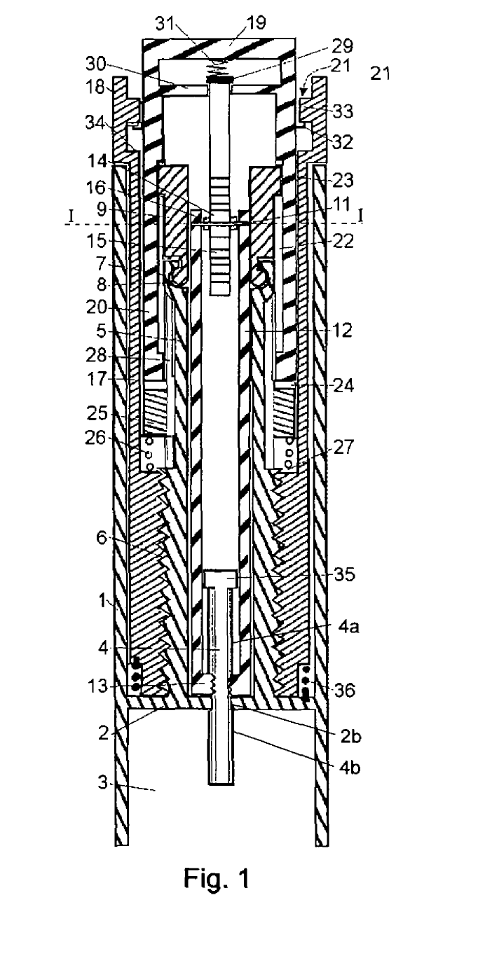

FIG. 1 schematically shows a sectional view of an injection device according to the invention, and

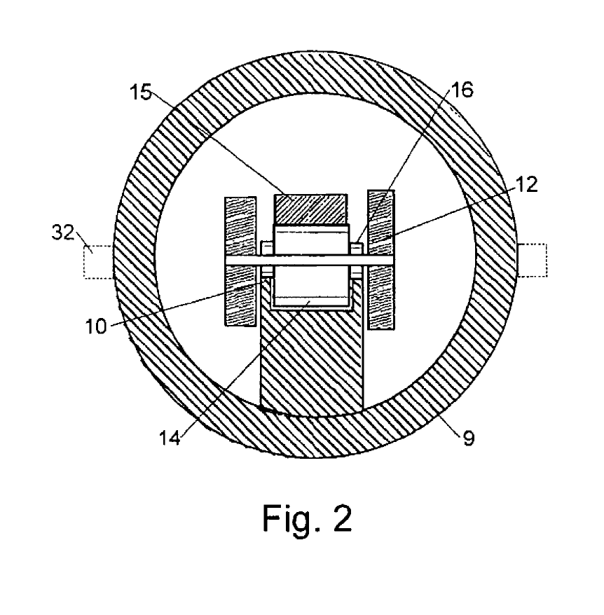

FIG. 2 shows schematically a sectional view of the gear box along the line I-I in FIG. 1,

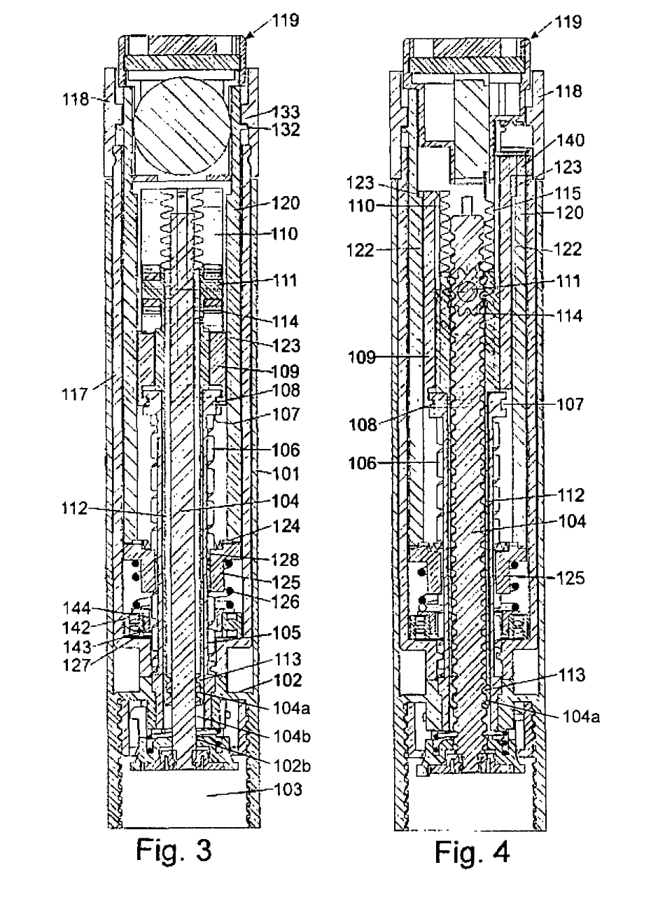

FIG. 3 shows a longitudinal sectional view in the dose setting part of another embodiment of an injection device according to the invention,

FIG. 4 shows a longitudinal sectional view perpendicular to the view in FIG. 3, and

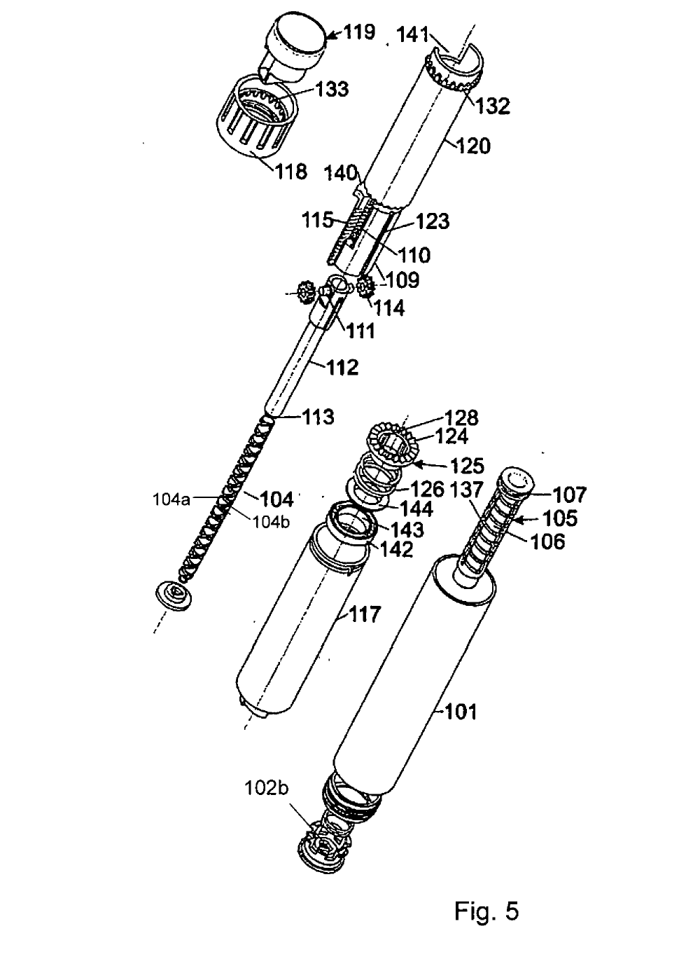

FIG. 5 shows an exploded view of the device shown in FIGS. 3 and 4.

The device shown in the appended drawings is shown as an injector pen, which pen has an elongated body with a central axis. However, other forms are within the scope of the invention.

In the device shown in FIG. 1 an elongated cylindrical housing 1 has a partitioning wall 2 which divides the housing in a compartment containing a dose setting mechanism and a compartment 3 designed for the accommodation of a not shown ampoule. The partitioning wall 2 is provided with a threaded opening 2b extending there through. Alternatively, the wall may be replaced by an insert which is secured against rotational and axial motion with respect to the main structure of the housing 1, the threaded opening 2b being formed in said insert. A piston rod 4 can be displaced longitudinally through the threaded opening 2b. The piston rod 4 has a first thread 4a and an additional longitudinally extending track forming a second thread 4b, wherein the second thread 4b mates with the threaded opening 2b.

Hence, when the piston rod is axially displaced, it is forced to rotate in accordance with the second thread 4b.

In accordance with the specific design of the injection device, the first thread 4a and the additional longitudinally extending track 4b may be disposed on opposite ends of the piston rod 4, may be partly overlapping or fully overlapping in the axial direction of the piston rod 4. Furthermore, the thread 4a may be disposed with a larger depth than the additional longitudinally extending track 4b or vice versa. In FIG. 1, the threads 4a and 4b and the threaded opening 2b are schematically represented.

Concentrically with the housing 1 the wall 2 carries on its side turning away from the compartment 3 a tubular element 5 which is at a part of it adjacent to the wall 2 provided with an outer thread 6 and which has at its free end a circumferential recess 7. A ring shaped coupling element 8 on a gearbox 9 engages the recess 7. By this coupling the gearbox is fixed in the housing 1 in a way that allows the gearbox 9 to rotate in the housing but not to be axially displaced relative to said housing.

In the gearbox 9 a gear wheel assembly comprising two integral gear wheels is journaled on a shaft 11, which runs perpendicular to the longitudinal axis of the device between two axial connection bars 12. The connection bars 12 project from the gearbox towards the partition wall 2 and are connected to a nut 13 which engages the first thread 4a of the piston rod 4. The gear wheel assembly comprises a gear wheel 14 with a large diameter engaging the teeth of a rack 15 which is guided in the gear box to be displaced in the longitudinal direction of the device, and a gear wheel 16 with a small diameter engaging a rack 10 in FIG. 2 extending in the longitudinal direction of the device on the inner wall of the gearbox 9. The gear wheel 16 with the small diameter may be divided into two gear wheels placed on each side of the of the gear wheel 14, and the rack on the inner wall of the gearbox 9 may have a longitudinal recess without any teeth to make room for the gear wheel 14.

A tubular dose setting member 17 fitting into the housing 2 is at an end provided with an internal thread mating and engaging the outer thread 6 of the tubular element 5 and has at its other end a part with enlarged diameter forming a dose setting button 18. Due to the engagement with the thread 6 the dose setting member 17 may be screwed in and out of the housing to show a number on a not shown helical scale on its outer surface in a not shown window in the housing 1.

A bottom 19 in a deep cup shaped element, which has a tubular part 20 fitting into the dose setting member 17 and encompassing the gearbox 9, forms an injection button. Coupling means between the dose setting member 17 and the cup shaped element ensures that rotation of the dose setting member 17 is transmitted to the cup shaped element. Further the inner wall of the tubular part 20 has longitudinal recesses 22 engaged by protrusions 23 on the gearbox 9 so that rotation of the dose setting member 17 via the cup shaped element is transmitted to the gearbox 9.

At the edge of the open end of the cup shaped element a rosette of V-shaped teeth are provided, which teeth engage a corresponding rosette of V-shaped teeth 24 on a coupling member 25 which is pressed against the edge of the cup shaped element by a spring 26 which is compressed between a not toothed side of the coupling member 25 and a round going shoulder 27 on the inner wall of the dose setting member 17 at an inner end of the inner thread of this member. The coupling member 25 is provided with an inner recess, which is engaged by a longitudinal rib 28 on the tubular element 5 so that the coupling member 25 can be displaced in the axial direction of the device but cannot be rotated relative to the housing 1. Thereby a click coupling is established which makes a click noise when the V-shaped teeth at the edge of the cup shaped element by rotation of this element rides over the V-shaped teeth of the coupling member 25.

A head 29 on the projecting end of the rack 15 is with a play fixed at the bottom of the cup shaped element between the bottom 19 forming the injection button and an inner wall 30 near this bottom. The rack is fixed in a position with its head pressed against the wall 30 by a spring 31 between the bottom 19 and the head 29.

To set a dose the dose setting button 18 is rotated to advance the dose-setting member 17 in the proximal direction in accordance with the thread 6. Due to the coupling 21 the cup shaped element will follow the rotation of the dose-setting member 17 and will be lifted concurrently with this member up from the end of the housing 1. During dose setting, the respective amount of axial displacement of the dose setting member 17 and the injection button 19 depends on the pitch of the thread 6, the pitch of the first thread 4a on the piston rod 4, and the gear ratio. Also, if the piston rod 4 is allowed to move during dose setting, the axial and rotational movements of the piston rod 4 influence the amount that the injection button will be displaced. By the rotation of the cup shaped element the V-shaped teeth 24 at the edge of its open end will ride over the V-shaped teeth of the non rotatable coupling member 25 to make a click sound for each unit the dose is changed. A too high set dose can be reduced by rotating the dose setting button 18 in the opposite direction of the direction for increasing the dose. When the dose setting member is advanced in the proximal direction the coupling member 25 will follow the dose setting member 17 in its axial movement as the spring 26 is supported on the shoulder 27. The spring will keep the V-shaped teeth of the coupling member 25 and the cup shaped element in engagement and maintain in engagement the coupling 21, which may comprise Δ-shaped protrusions 32 on the cup shaped element engaging ̂-shaped recesses in an inner ring 33 in the dose setting button 18.

The rotation of the dose setting button 18 and the cup shaped element is further transmitted to the gearbox 9 through the protrusions 23 on this gearbox engaging the longitudinal recesses 22 in the inner wall of the tubular part 20 of said cup shaped element. The rotation of the gearbox 9 is through the connection bars 12 transmitted to the nut 13, which in this way is advanced along the first thread 4a of the piston rod 4 and lifted away from its abutment with the wall 2 when a dose it set. As the dose is set by moving the nut 13 on the piston rod which operates the piston in the not shown ampoule in the compartment 3, a dose setting limiter, which ensures that the size of the set dose does not exceed the amount of medicament left in the ampoule, can easily be established by providing the piston rod 4 with a stop 35 which limits the movement of the nut 13 up along the piston rod 4.

Due to the confinement of the head 29 in the space between the bottom 19 and the wall 30 of the cup shaped element, the rack 15 is drawn with the injection button outward. Depending on the specific gear ratio of the gear assembly, the outward movement of the rack 15 corresponds to an outward movement of nut 13. As the nut 13 is rotated during dose setting, the length of the piston rod/nut assembly is prolonged, depending on the mutual pitch of the piston rod/nut threads and depending on the actual design of the additional longitudinally extending track 4b. By varying the pitch of the different threads or the gear ratio, the piston rod is either moved distally, proximally or held stationary during dose setting.

Ideally, if no axial movement of the piston rod 4 is wanted during dose setting the pitch of the thread 6 and the thread formed in the piston rod 4a as well as the pitch of the outer recess (6) should be matched exactly to the gearing ratio of the gear box depicted in FIG. 2.

However, tolerances in the machining of the components comprising the pen may lead to a slight unintended mismatch of the pitches relatively to the transmission ratio of the gearbox 9. Especially the situation where the piston rod 4 advances slightly during dose setting is highly undesirable as the injection device during dose setting will expel a small amount of medication.

This situation is avoided by mismatching one or more of the above mentioned pitches to the transmission ratio of the gearbox such that the piston rod 4 is slightly withdrawn during dose setting. The mismatch of pitches is most simply implemented by modification of the pitch of the recess 4a made in the piston rod 4 and the corresponding thread in the nut 13. In addition, if the additional longitudinally extending track 4b on piston rod forms a helical thread, i.e. allowing the piston rod 4 to rotate with respect to the housing (1), this threaded connection has to be taken into account when designing the mechanism.

Alternatively, the injection device is designed such that the recess in the piston rod is initially designed to be axially aligned with the piston rod. On assembled production prototypes the advance of the piston rod during dose setting is measured. Hereafter the axial aligned recess is redesigned to a helical recess where the superimposed rotation of the piston rod during dose setting compensates for the tolerances in the machining.

According to one embodiment of the invention the pitches of the threaded parts and the linear gearing are matched such that the piston rod retracts between 0% and 2% of the full linear translation relatively to the cartridge during dose setting.

A corresponding solution can be provided in the injection device shown in U.S. Pat. No. 6,663,602 which contains an injection device having a coupling member 25, which is kept rotationally locked with respect to the housing. For that particular injection device, a slight mismatch of either the thread 6 or the thread of piston rod 4 to the transmission ratio of the gearbox can be provided such that the piston rod 4 is slightly withdrawn during dose setting.

To inject a set dose the injection button is pressed by pressing on the bottom 19. In the initial phase of the pressing the spring 31 is compressed where after the pressing force is directly transmitted to the head 29 of the rack 15 and this way to the rack 15 itself. Through the gear box 9 the force is transformed and is transmitted through the connection bars 12 to the nut 13 which will press the piston rod 4 into the compartment 3 until the dose-setting member 17 abuts the wall 2. During advance of the piston rod 4, the piston rod will rotate in accordance with the additional longitudinally extending track of the piston rod 4. Hence, the resulting displacement during dosing will depend on both the actual displacement of the nut 13 and also the displacement of the piston rod which occurs due to its threaded engagement with the housing 1.

During the initial phase of the movement of the injection button the Δ-shaped protrusions 32 on the cup shaped element will be drawn out of their engagement with the ̂-shaped recesses in the ring 33. The dose-setting member 17 can now rotate relative to the injection button and will do so when the Δ-shaped protrusions 32 press against a shoulder 34 at the bottom of the dose setting button 18. Only a force sufficient to make the dose setting member rotate to screw itself downward along the thread 6 is necessary as the force necessary to make the injection is transmitted to the piston rod 4 through the gearbox 9. A helical reset spring 36 concentric with the dose setting member can be mounted at the lower end of this member and can have one end anchored in the dose setting member 17 and the other end anchored in the wall 2. During setting of a dose this spring may be tighter coiled so that on the dose setting member it exerts a torque approximately corresponding to the torque necessary to overcome the friction in the movement of the dose setting member along the thread 6 so that the force which the user have to exert on the injection button is only the force necessary to drive the piston rod into an ampoule to inject the set dose.

It shall be noticed that use of only one size gear wheel which engages as well the rack 15, which is movable relative to the gear box 9, as the rack 10, which is unmovable relative to the gear box, provides a gearing ratio of 2:1 for the longitudinal movement relative to the syringe housing 1 for the movable rack 15 and the connector 12, which carries the shaft 11 of the gear wheel.

FIGS. 3 and 4 shows a preferred embodiment wherein only one size gear wheel is used and wherein elements corresponding to elements in FIGS. 1 and 2 are given the same references as these elements with a prefixed “1”.

For manufacturing reasons minor changes are made. So the partitioning wall 102 and the tubular element 105 are made as two parts which are by the assembling of the device connected to each other to make the assembled parts act as one integral part. The same way the dose setting member 117 and the dose setting button 118 are made as two parts, which are fixed firmly together.

A circumferential recess 107 is provided as an outer recess at the free end of the tubular part 105 and a ring shaped coupling element is provided as an inner bead 108 on the gearbox element 109 which bead engages the recess 107 to provide a rotatable but not axially displaceable connection between the tubular part 105 and the gearbox.

A tubular element 120 having ridges 122 which engages recesses 123 on the gearbox is at its upper end closed by a button 119 from which a force provided by pressing this button is transmitted to the tubular element 120.

The gearbox is formed by two shells, which together form a cylinder fitting into the tubular element where the shells are guided by the engagement between the ridges 122 and the recesses 123. Racks 110 and 115 are provided along edges of the shells facing each other. One shell forming the gearbox part 109 is provided with the inner bead 108, which engages the circumferential recess 107 at the end of the central tubular part 105 and carries the rack 110. The other shell is axially displaceable in the tubular element 120 and forms the rack 115. At its outer end projecting from the gearbox the shell carrying the rack 115 is provided with a flange 140 which is positioned in a cut out 141 in the end of the tubular element 120 carrying the button 119 so that this button and the tubular element 120 can be moved so far inward in the device that the engagement of the teeth 132 and 133 can be released before the button 119 abuts the flange 140.

A tubular connection element 112 connects the threaded piston rod 104 with the gearbox. At its end engaging the piston rod 104 the connection element has a nut 113 with an internal thread mating the external thread of the piston rod. At its end engaging the gear box the connection element is provided with two pins 111 projecting perpendicular to the longitudinal axis of the connection element 112 at each side of this element. Each pin 111 carries a gear wheel 114 which is placed between and engages the two racks 110 and 115. This way the connection element 112 will be rotated with the gear box but can be displaced axially relative to said gear box when the racks 110 and 115 are moved relative to each other. In practice it will be the rack 115, which is moved relative to the gearbox element 109 and the housing and will by the shown construction result in a movement of the connection element 112 relative to housing a distance which is half the distance which the rack 115 is moved. A coupling member 125 which is at its periphery provided with a rosette of teeth 124 and has a central bore fitting over the central tube in the housing 101 so that this coupling member 125 can be axially displaced along said central tube 105, but internal ridges 128 in the central bore of the coupling member 125 engages longitudinal recesses 137 in the central tube to make the coupling member non rotatable in the housing so that a rosette of teeth at the edge of the tubular element 120 can click over the teeth 124 of the coupling member when said tubular element is rotated together with the dose setting member 117. A spring 126 working between the coupling member 125 and an internal shoulder 127 provided in the dose setting member 117 makes the coupling member follow the tubular element 120 when this element with the dose setting member is moved longitudinally in the housing. To make the dose setting member easy rotatable, especially when said dose setting member is pressed inward in the housing, a roller bearing having an outer ring 142 supported by the shoulder 127 and an inner ring 143 supporting a pressure bushing 144 which supports the spring 126. By the provision of this smooth running support only very small axial forces are needed to rotate the dose setting member 117 back to its zero position when a set dose is injected. This solution replaces the provision of a reset spring as the spring 36 in FIG. 1. The bearing is shown as a radial bearing but can be replaced by an axial bearing.

As discussed in relation to the embodiment shown on FIG. 1 and 2, the piston rod has a first thread for engaging the threaded part of the nut and an additional longitudinally extending track 104b for engaging the threaded opening of partition wall. FIG. 5 shows an exploded view of the embodiment of FIGS. 3 and 4, the partition wall 102 being equipped with a separate insert member 102b being fixedly mounted with respect to the housing. Insert member 102b is provided with a threaded opening to receive and engage the additional longitudinally extending track 104b of the piston rod 104.

Although the above described embodiments show different specific gearbox assemblies, it is to be noted that, within the scope of the present invention, various other types of gearboxes can be used.

In addition, alternative embodiments having a dose setting member which is kept axially fixed during dose setting/dosing will be applicable with the present invention and within the scope of the appended claims. One non-limiting embodiment comprises an additional slave member which is rotationally coupled to the dose setting member, and which can be used for inducing axial movement on the coupling member during dose setting.

Further, a frictional coupling may be provided instead of the above-described incremental feedback mechanism, whereby doses may be set among a continuous selection of dose amounts.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

1. An injection device comprising:
a. a housing having a proximal end and a distal end,
b. a threaded piston rod having a first thread with a first pitch and a second thread with a second pitch, the piston rod being linearly displaceable in the housing,
c. a member secured against rotational and axial motion with respect to the housing the member forming a central threaded opening for engaging the second thread of the piston rod,
d. a displaceable nut that moves relative to the housing the nut engaging the first thread of the piston rod so that the nut can screw along the first thread of the piston rod, thereby being capable of moving along the piston rod from a first position on the piston rod to a second position on the piston rod during dose setting, and from the second to a third position during dosing, the displacement of the nut along the piston rod from the first position to the third position defining the quantity of medication expelled by the injection device,
e. a gearbox assembly coupling linear displacement of an injection button to axial displacement of the nut, and
f. means for preventing rotation of the nut when linear force is exerted on the injection button.

2. The injection device as defined in claim 1, wherein the gearbox assembly causes the nut to move a distance in a distal direction during injection that is less than the distance moved by the injection button.

3. The injection device as defined in claim 1, wherein the gearbox assembly comprises at least one gear wheel and racks in permanent engagement.

4. The injection device as defined in claim 1, wherein the device further comprises a dose setting member coupled to the nut, the dose setting member protruding from the proximal end of the housing and having an injection button located on a proximal end thereof, the injection button adapted to receive a linear force.

5. The injection device as defined in claim 3, wherein the dose setting member during dose setting is rotationally coupled to the nut so that rotation of the dose setting member causes the nut to screw along the piston rod.

6. The injection device as defined in claim 1, wherein the first thread and the second thread on the piston rod are oppositely disposed.

7. The injection device as defined in claim 1, wherein the pitches of the threaded parts and the linear gearing are matched such that the piston rod retracts slightly relatively to the cartridge during dose setting.

8. The injection device as defined in claim 2, wherein the gearbox assembly comprises at least one gear wheel and racks in permanent engagement.

9. The injection device as defined in claim 2, wherein the device further comprises a dose setting member coupled to the nut, the dose setting member protruding from the proximal end of the housing and having an injection button located on a proximal end thereof, the injection button adapted to receive a linear force.

10. The injection device as defined in claim 3, wherein the device further comprises a dose setting member coupled to the nut, the dose setting member protruding from the proximal end of the housing and having an injection button located on a proximal end thereof, the injection button adapted to receive a linear force.

11. The injection device as defined in claim 2, wherein the first thread and the second thread on the piston rod are oppositely disposed.

12. The injection device as defined in claim 3, wherein the first thread and the second thread on the piston rod are oppositely disposed.

13. The injection device as defined in claim 4, wherein the first thread and the second thread on the piston rod are oppositely disposed.

14. The injection device as defined in claim 5, wherein the first thread and the second thread on the piston rod are oppositely disposed.

15. The injection device as defined in claim 2, wherein the pitches of the threaded parts and the linear gearing are matched such that the piston rod retracts slightly relatively to the cartridge during dose setting.

16. The injection device as defined in claim 3, wherein the pitches of the threaded parts and the linear gearing are matched such that the piston rod retracts slightly relatively to the cartridge during dose setting.

17. The injection device as defined in claim 4, wherein the pitches of the threaded parts and the linear gearing are matched such that the piston rod retracts slightly relatively to the cartridge during dose setting.

18. The injection device as defined in claim 5, wherein the pitches of the threaded parts and the linear gearing are matched such that the piston rod retracts slightly relatively to the cartridge during dose setting.

19. The injection device as defined in claim 6, wherein the pitches of the threaded parts and the linear gearing are matched such that the piston rod retracts slightly relatively to the cartridge during dose setting.
